(19) United States
(12) Patent Application Publication
(10) Pub. No.: US 2006/0167367 A1
(43) Pub. Date: Jul. 27, 2006

(54) Method and system for collecting data on a plurality of patients

(76) Inventors: James Stanczak, Chicago, IL (US); Russell Deremer, Midlothian, VA (US)

Correspondence Address: BARNES & THORNBURG, 11 SOUTH MERIDIAN, INDIANAPOLIS, IN, 46204, US

(21) Appl. No.: 11/313,491

(22) Filed: Dec. 21, 2005

Related U.S. Application Data
(60) Provisional application No. 60/639,017, filed on Dec. 23, 2004.

(51) Int. Cl. A61B 5/04

(52) U.S. Cl. 600/523

(57) ABSTRACT

A system and a method for recording data from one or more persons is provided. The system generally comprises a recording device for recording data from each person, a processing unit for storing and analyzing the recorded data, and one or more output devices for displaying or printing the data and/or the results of the analysis. The method provides steps for recording and analyzing the data from the one or more persons.

This application claims priority to and the benefit of U.S. Provisional Patent Application No. 60/639,017, filed on Dec. 23, 2004, the disclosure of which is now expressly incorporated herein by reference.

### FIELD OF THE INVENTION

This invention relates to the field of collecting data from a person and more specifically to collecting physiological data from a plurality of persons.

### BACKGROUND OF THE INVENTION

The Food and Drug Administration (FDA) desires that all drugs that make it into a person's bloodstream undergo cardiac safety testing (CST) in clinical trials to ensure that they do not cause sudden cardiac death or other cardiac complications. These trials, tests or studies typically comprise four defined phases. On average, about 80,000 people volunteer to participate in about 5,000 to 6,000 Phase 1 trials and about 3,000 to 4,000 Phase 2 trials conducted each year in conjunction with new drug applications. Such clinical trials are conducted pursuant to a defined test protocol by any one of about 900 or more Clinical Research Organizations (CRO) and/or 130 or more pharmaceutical companies. Illustratively, the test protocol may define such things as the drug to be tested; the frequency and dosage or titration of the drug to be taken by patients in the study; the duration of the study; when each patient should perform a certain event, such as lying down in order to record a resting ECG; and the like.

One focus of CST is to determine whether the drug has a propensity to create fatal cardiac arrhythmias, which may be indicated by a prolonged QT interval. A person's QT interval can be measured or determined from an electrocardiogram (ECG), which depicts a person's heart rhythm. The FDA desires that ECG data from these studies be submitted electronically, for example in extended mark-up language (XML). The FDA requires that all technology used to collect and analyze the data from the clinical studies be in compliance with 21 C.F.R. Part 11 as it relates to security and audit trails. For example, any changes to the QT intervals made during the analysis of an ECG must be tracked to create an audit trail.

During Phase 1 clinical trials, on average 20 to 100 persons or patients to be tested come to a centralized laboratory for one or more days of testing. These patients are volunteers who are not known to be suffering from the ailment targeted by the drug being studied or tested. During this testing, technicians typically use a conventional cardiograph, typically one per every three to four volunteers, to take up to twenty-ECGs-per day over multiple three-to-five-day periods. Generally, it requires three technicians, one per 8-hour shift over a 24-hour period, to run each of the cardiographs. Because there typically is only one cardiograph and one technician for every three or four volunteers, the 12-ECG recording is not continuous. Rather, each technician must rotate the cardiograph between each of the three to four volunteers or patients assigned to that cardiograph. This manual administration of ECGs may lead to inconsistent timing of ECGs associated with drug titrations as the technicians rotate the cardiograph from one patient to the next. Generally, these conventional cardiographs do not have a screen to display the ECGs as they are being taken, and do not provide immediate alerts in the event of an ECG associated with adverse reaction. Rather, each 12-lead ECG is typically printed out on some type of thermal printer for a physician to read and measure manually. The study protocols and patient demographics assigned to each cardiograph are generally unsaved; and therefore must be entered manually on multiple occasions.

During Phase 2 testing, all cardiac drugs and all drugs shown during Phase 1 trials to have at least a marginal affect on cardiac safety, undergo further CST. Phase 2 involves the testing of the drug of interest on hundreds of patients who are known to be suffering from the ailment targeted by that drug. In contrast to the Phase 1 testing, which is conducted at a central location, this Phase 2 testing, which may last for months or years, may be conducted throughout the world at remote sites. In accordance with the protocol, about half of the patients in these double-blind Phase 2 tests will actually receive the drug of interest, while the other half will receive a placebo. The patients undergo regular ECG testing while they are taking the drug. The clinicians involved in the Phase 2 studies may or may not be familiar with the operation of a standard cardiograph. Also, these standard cardiographs typically have a limited storage capability, perhaps as low as about ten seconds, perhaps leading to technically inadequate ECGs. These ECGs typically are sent from the remote site to a central location for manual analysis.

It is known to take periodic ECGs of patients involved in clinical drug studies. As noted above, these periodic ECGs generally are not taken at the same time for each patient. It is also known to record a person's physiological data, including a person's ECG, with portable devices. For example, a Holter system is known in the art as a portable device that collects ECG data from a person continuously over a period of time, usually a 24-hour period of normal activity for that person. It is desirable to have a system capable of recording data or information, such as ECG data, simultaneously from a plurality of persons. Such a system would be useful in gathering, storing and evaluating data from a plurality of persons involved in a study, including a clinical drug or pharmaceutical study.

### SUMMARY OF THE INVENTION

The present invention may comprise one or more of the features set out in the co-pended claims or the following features or combinations thereof. A system for gathering information on a plurality of persons is provided. The plurality of persons may be involved in a clinical study, such as a drug or pharmaceutical study. In pharmaceutical studies it is desirable to administer a drug to a person, or plurality of persons, and then measure and record for analysis certain physiological information, such as heart rhythm as seen on an ECG, of that person(s) during normal activity for a defined period. As used herein a person could be a volunteer, a subject, or a patient, and these terms are used interchangeably throughout this specification. As used herein, a user may be, for example and without limitation, any one or more than one of the following: a clinician, a doctor, a nurse, a technician, a patient, a test subject, volunteer, or any other person. It is further desired that the collected data be converted to an electronic format, such as for example XML. The disclosed system is usable in all phases, i.e., Phases 1-4, of pharmaceutical testing in humans. The system may also be used in clinical settings, such as doctors' offices, clinics or hospitals.

The specific characteristics of the disclosed collection system may vary generally, and may vary depending on whether the system is for clinical use or for pharmaceutical testing. If the system is being used for pharmaceutical testing, the characteristics may vary depending on which phase of testing is involved. For example, Phase 1 testing generally requires more robust recording devices and management tools than does Phase 2 testing. Similarly, the composition of a clinical system, not involved in pharmaceutical testing, may vary from the system(s) used in Phase 1 or Phase 2 testing. Generally speaking, however, each system illustratively may comprise generally one or more recording devices and a management device or system. Each recording device may be an ambulatory medical recording device (AMRD).

The management device or system may be control circuitry and associated software integral to each recording device and/or it may be one or more computers at a central site, or one or more computers at one or more remote sites, or any combination of central and remote site computers and recording device processors. If the management device is separate, in whole or in part, from the recording device(s), then each recording device may be connected with the management device, and vice versa, through any suitable wired or wireless communication protocol, connection or interface. No matter the number or location of central and/or remote computers, they may, but need not be networked with one or more of the other computers. In any event, the remote computers and the recording devices illustratively will be able to communicate with the central site computer(s) via a wired or wireless, two-way signal path or connection. The connection may be a secure connection. The computers can be any computer, including an industry standard computer running industry standard software, such as for example a personal computer running windows software.

The management device or system may manage various aspects of the data gathering, storage and analysis, including data download and database file creation. Illustratively, the management device used in pharmaceutical testing may initialize each AMRD simultaneously by transmitting to each AMRD for upload, the entire patient class demographics, including a guaranteed unique identification (GUID), as well as the test protocol. The test protocol may include start times, security options, a listing of patient events or symptoms, and alarm/patient/staff notifications. As noted, the management device also assigns a unique serial number to each of the patients and associates one serial number with a single AMRD per protocol. The AMRD and the management device may retain indefinitely, and retrieve, the study and class demographics and protocols. During the course of Phase 1 testing, the management system may receive ECGs transmitted over a wireless connection for immediate display and/or printing. This immediate transmission of ECG data may be initiated by a user, for example by actuating a transmit button on the AMRD, or may be initiated automatically by the software in response to a certain event or scheduled time. The ECG can also be observed at any time on the AMRD display. The management system may prescribe and collect from each AMRD, pre-programmed, timed ECG output so dosing schedules and titration timings are precise. The management software may provide for the confirmation of successful ECG transfers and may assure the avoidance of redundant transfers. The management system can also retrieve previous or baseline ECGs for comparison and can track changes to the data. Illustratively, the management system can automatically analyze the physiological data, including the ECG data, can provide tools for manual analysis of the ECG data and editing of the automatic analysis, and may interface with third party computers and tools for analysis and storage of data. The management system can also collect data from the AMRD entered by a user from a patient activated patient events and symptoms list. The management system used in Phase 2 testing, may be one or more remote site computers in communication with the central computer used during Phase 1 testing. The remote computer may transmit data to the central computer using standard information technology hardware and software, for example using the Internet and XML format. The remote computer may send data immediately or using a scheduling program for after-hours transmissions. The management system used in a clinical setting, such as a doctor's office or in a clinic, may comprise a non-server manager for communicating, printing, archiving, and editing ECGs from an AMRD. In this setting, a plurality of AMRDs is not required. Such a clinical system may include software to allow processing of data not only in XML format, but also may be compatible with International Society of Holter and Noninvasive Electrocardiology (ISHNE) protocols or standards for collection, storage and transmission of data from Holter analyzers. The system may also receive data from exercise stress testing in .pdf or other formats and may integrate to electronic medical record (EMR) or hospital information systems (HIS) with custom or HL7 standard communications links.

Each recording device may be an ambulatory medical recording device (AMRD) and may have one or more sensors connected to, coupled to, or in communication with the recording device via a wireless or a wired connection. The sensor(s) may be electrodes, which may be of any suitable number and type, including suction or adhesive electrodes. These electrodes are placed on the body of a person in order to measure the person's ECG. The person may be one of a plurality of persons participating in a drug or pharmaceutical test or study pursuant to a defined test protocol. A separate AMRD may be provided for each of the plurality of persons in the test, with each patient being assigned a unique identification or serial number. Each AMRD may have a sampling rate of up to and including about 1,000 Hz or more. Each AMRD may record data gathered by the sensor(s) and may output this data to a display, a printer, or a database for storage, analysis and/or review. For example, each AMRD may have a display to review the data gathered by the sensor(s). Such a display may be a black-and-white display, a gray-scale display, or a liquid crystal display (LCD) and may display text, data, graphical and pictorial information to a user. The graphical display may be able to display electrocardiograms (ECG), including three-lead, six-lead, and twelve-lead ECGs. Each AMRD may also output the data to a printer. Such a printer may be integral to the AMRD, may be connected to the AMRD directly by a wired or wireless connection, or may be connected to a central site or remote computer which in turn is connected to each AMRD through a wired or wireless connection. The printer may be a thermal, laser, inkjet, impact or other suitable printer. Each AMRD may further comprise any suitable permanent and/or removable storage media and may be capable of continuous recording of ECGs in excess of ten seconds and illustratively for a minimum of about 10 minutes up to about 24 hours or more. Illustratively, each patient and test protocol will have a GUID assigned by the management device allowing any one AMRD to store and retain indefinitely multiple test protocols and the demographics and test results of multiple patients. For example, any one AMRD may have a first protocol loaded and be associated with a first patient enrolled in that first protocol. That same AMRD also may be loaded with a second protocol and associated with a second patient, or even that same first patient, enrolled in that second protocol. A patient or other user may interface with an AMRD using for example one or more keys or system navigation buttons. Such interface may be governed by one or more security devices or protocols. The patient may enter symptoms manually or by using the keys to select from a list of patient events or symptoms. The AMRD may come in different sizes and configurations depending on the desired use. For example, a Phase 1 AMRD may range in size from about 4 to about 8 inches and about one-half to one inch in thickness, whereas a Phase 2 AMRD may range in size from about 3 to about 6 inches and about one-quarter to three-quarters of an inch in thickness. A Phase 1 AMRD may use rechargeable batteries, whereas a Phase 2 AMRD might use disposable batteries. Further illustratively, in the case of AMRDs used in Phase 1 trials, each AMRD might be able to record continuously for about 24 hours or more. In the case of an AMRD used in a Phase 2 trial, the AMRD might be able to record in excess of ten seconds of data, up to about 24 hours of data.

As noted the collection system illustratively will support standard communication protocols such as for example XML and/or ISHNE protocols. The system may print, edit, store, archive, transmit and export in electronic format such as XML. Therefore, the test data may be downloaded via a wired or wireless connection for electronic transmission to one or more recipients in XML, for example using the Internet. Such recipients may include for example and without limitation the FDA, a CRO, a pharmaceutical company, a physician, a clinician and the like. The system, including each AMRD and management device, illustratively will provide for secure access and will comply with 21 C.F.R. 11, including security and audit trail requirements, as well as the Health Insurance Patient Portability Act (HIPPA) and other applicable standards, rules, regulations and laws. All electronic transmissions of patient information may be encrypted for security. The management system may collect and transmit ECGs and other data using any suitable encoding protocol, for example 128-bit encryption.

Exhibits 1 and 2 attached hereto provide further background and details of the invention, including details of an illustrative storage file. These and other aspects of the present invention will become more apparent from the following description of the illustrative embodiment.

### BRIEF DESCRIPTION OF THE DRAWINGS

FIG. 1 is a diagrammatic illustration of a system used for collecting data on one or more patients.

FIG. 2 is a diagrammatic illustration of a general purpose computer system operable within the illustrative collection system of FIG. 1.

FIG. 3 depicts an illustrative monitoring device operable within the illustrative collection system of FIG. 1.

FIG. 4 depicts an illustrative monitoring device operable within the illustrative collection system of FIG. 1.

FIG. 5 depicts an illustrative monitoring device operable within the illustrative collection system of FIG. 1.

FIG. 6 is a flow chart depicting an illustrative method of collecting data on one or more patients.

FIG. 7 depicts an illustrative monitoring device operable within the illustrative collection system of FIG. 1.

FIG. 8 depicts an illustrative monitoring device operable within the illustrative collection system of FIG. 1.

### DESCRIPTION OF THE ILLUSTRATIVE EMBODIMENTS

For the purposes of promoting an understanding of the principles of the invention, reference will now be made to a number of illustrative embodiments illustrated in the drawings and specific language will be used to describe the same.

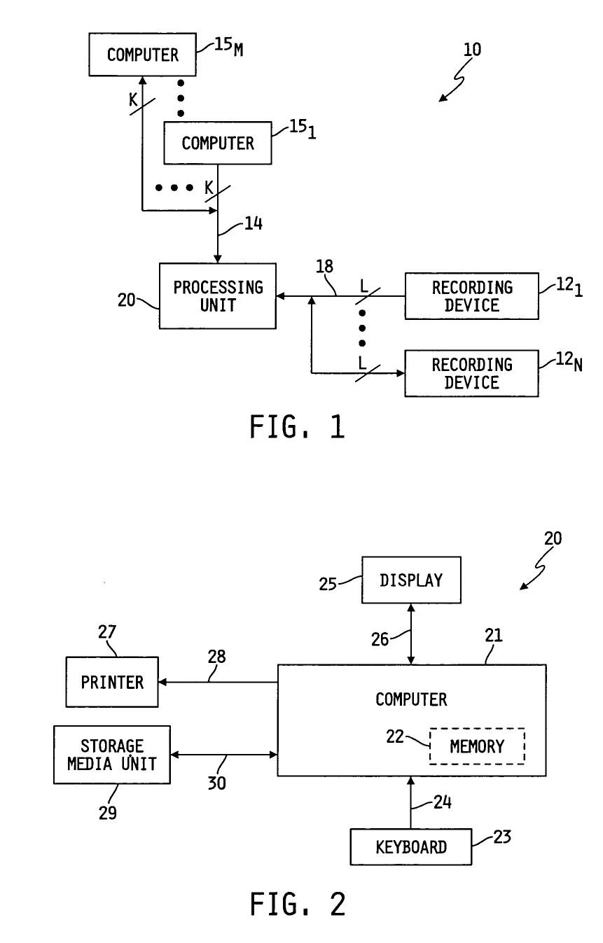

Referring now to FIG. 1, there is shown an illustrative system 10 for gathering patient data. Illustratively, the system 10 generally comprises a plurality of recording devices 12₁ through 12_{N}, where N is any positive integer, in two-way communication with one or more processing systems or units 20 through a number, L, of signal paths 18, where L may be any positive integer. The system 10 optionally may comprise one or more computer(s) or computer systems 15₁ through 15_{M}, where M may be any positive integer, which would also be in communication with the one or more processing systems 20 through a number, K, of signal paths 14, where K may be any positive integer. The signal paths L, K may comprise alone or in combination any suitable signal path or communications medium, standard, protocol or network such as for example and without limitation, alone or in combination, a hardwire, a radio frequency signal, or a light signal. Some illustrative mediums, standards, protocols or networks include without limitation, hardwire, Universal Serial Bus (USB), FireWire, i.Link, IEEE 1394, ethernet, cable modem, broadband DSL, the Internet, the Public Switched Telephone Network (PSTN), intranets, Local Area Networks (LAN), Wide Area Networks (WAN), Wireless Area Network or Wireless Local Area Network (WLAN) or any other suitable communication standard, system, standard or protocol such as for example and without limitation any Wireless Fidelity (Wi-Fi) system or network, including for example and without limitation 802.11a, 802.11b, and 802.11g Wi-Fi systems, bluetooth systems, infrared systems, and the like. The signal paths K, L may be separate or switched. The other computer(s) 15 may be any suitable computer or computers including a personal computer or a system of networked computers.

The plurality of recording devices 12₁ to 12_{N} may comprise any suitable ambulatory medical recording device (AMRD). The AMRD 12 illustratively comprises a memory device (not shown), a display 31, a power source (not shown), an audio output, and one or more communication devices or systems (not shown). The AMRD illustratively has an adjustable resolution and may utilize sampling rates of up to about 1,000 Hz. The power source illustratively comprises a battery (not shown). The battery may be rechargeable and interchangeable with one or more other batteries. The batteries may be changed out, or “hot swapped,” during recording to allow for multi-day continuous recording. So too, the AMRD may be equipped with a power chord in order to use AC power when desired. An illustrative AMRD may measure about 3 to 6 inches by about 4 to 8 inches and may be about one-half inches to about one inch in width. In one embodiment, the AMRD may measure about 4.25 inches by about 6 inches and may be about three-quarters of an inch in width. In another embodiment, the AMRD may measure about 3 inches by about 4 inches.

The memory device or medium (not shown) may be any suitable computing memory device, which may but need not be removable. A non-exhaustive example of suitable memory devices include without limitation, alone or in combination with one another, a chip, a disk, a hard drive, a tape, or a card as known to those skilled in the art. Some suitable examples include without limitation random access memory (RAM), read only memory (ROM), video memory card such as Video Random Access memory (VRAM), flash memory, and/or any suitable removable recording media such as a floppy disk, a compact disk (CD), a digital video disk (DVD), or a thumb drive, flash drive or memory stick. Illustratively the memory device is capable of continuous recording of electrocardiogram (ECG) information for a minimum of 24 hours, such as for example and without limitation a 12-lead ECG. In order to record any ECG the AMRD is in communication with electrodes placed on the body of a patient. The electrodes may be any suitable number and type of electrode including without limitation suction or adhesive electrodes. The electrodes and the AMRD 12 may be connected by one or more suitable communication pathways including without limitation hardwire 39, radio frequency signals, or light signals. The AMRD may further comprise an analog-to-digital converter in order to convert the signals from the electrodes to the graphical ECG and back to a digital signal for transmission to an external computer 15, 21.

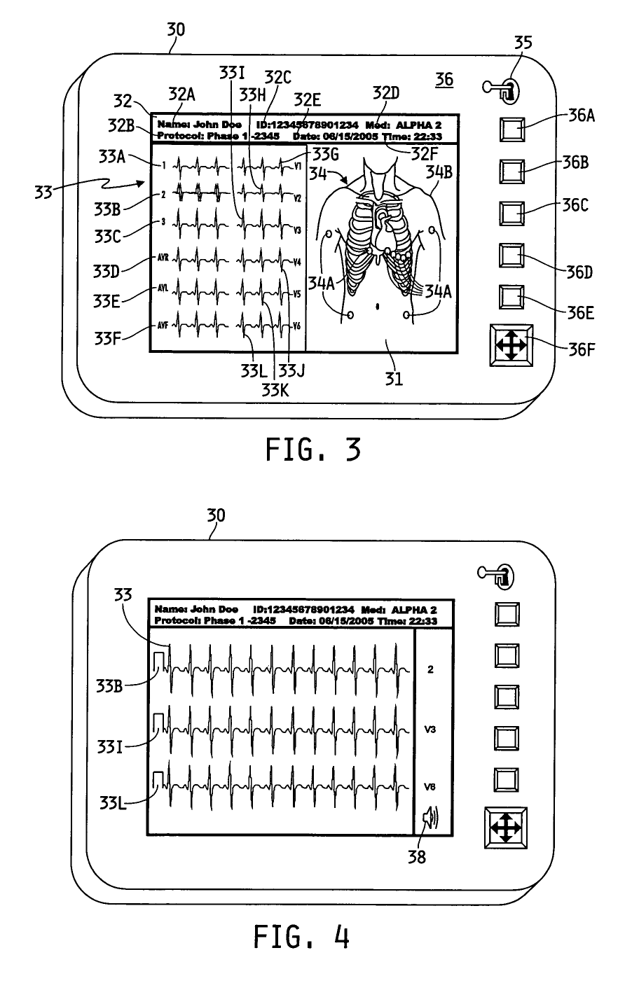

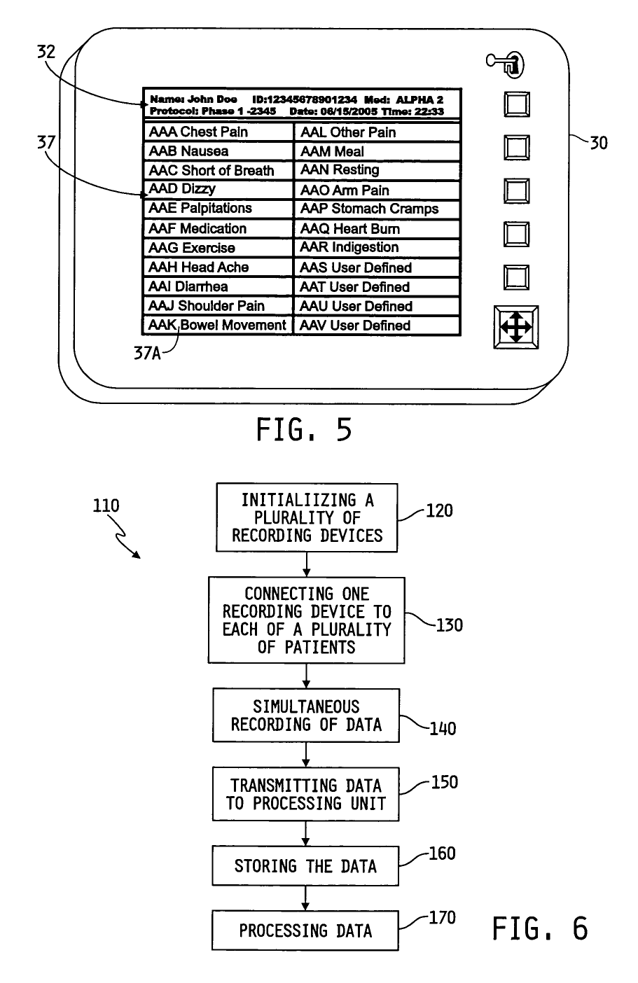

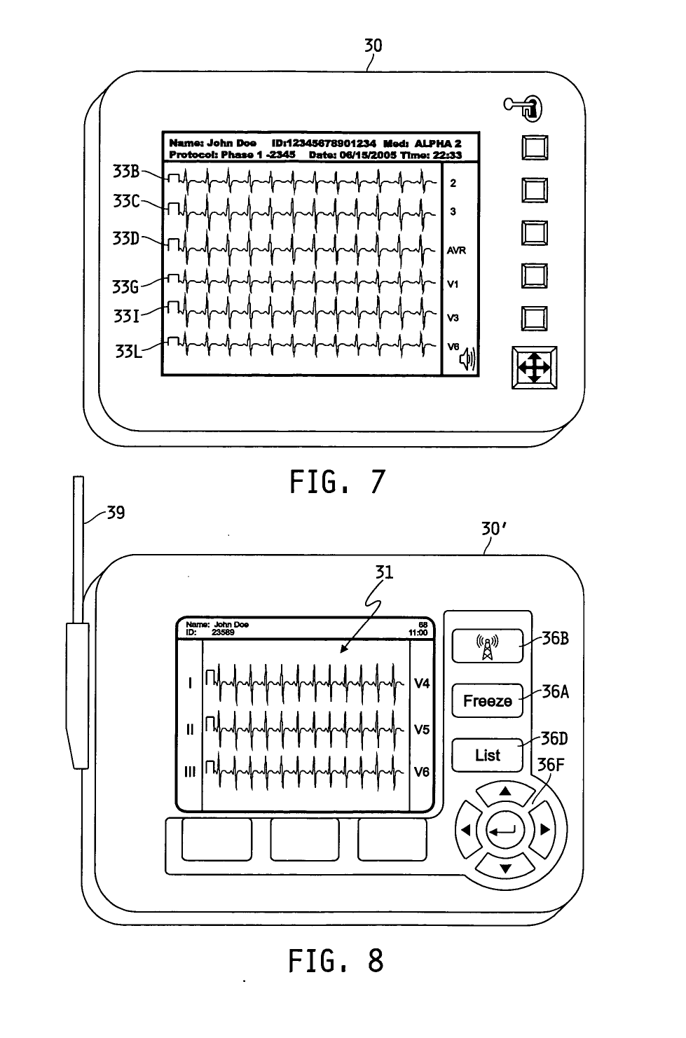

Referring to FIGS. 3 and 4, the AMRD display 31 illustratively may be a black and white, gray-scale, or color liquid crystal display (LCD) comprising a text display area 32, an ECG display area 33, and an electrode status display area 34. Any other suitable display may be used. The text display area 32 may display any desired information, for example and without limitation, it may display a patient's name 32A and identification number 32C, the name of the protocol 32B, the date 32D and time 32F of the displayed information and the medicine being evaluated 32E. Any other desired text could be displayed, either simultaneously or as selected by a user of the device. As used herein, a user may be, for example and without limitation, any one or more than one of the following: a clinician, a doctor, a nurse, a technician, a patient, a test subject, volunteer, or any other person. As used herein, a patient includes a test subject or volunteer, and does not necessarily connote a person who is ill or in need of medical treatment. The AMRD is capable of recording and displaying multiple standard ECG formats, as well as other graphical and text data or information as will be explained. For example and without limitation, the AMRD can display for review the 12-lead format 33A-L (FIG. 3), a 6-lead format 33B-D, G, I, L (FIG. 7), a 3-lead format 33B, I, L (FIGS. 4 and 8), or a freeze format. As seen in FIG. 5, text can be displayed in this area 33. The status display area 34 illustratively includes a graphical representation of the electrode placements 34A on a torso 34B. Depicted in FIG. 3 is a standard placement of ten electrodes 34A in a 12-lead configuration. Illustratively, further graphical, pictorial, and textual information could be displayed in this area 34 as seen for example in FIG. 5. The AMRD illustratively is equipped with a security key 35, for example and without limitation a USB hard lock activation key user identification and lock system or a radio frequency identification (RFID) proximity detector user identification and lock system. Illustratively, the AMRD also includes a control panel 36 with various control buttons, keys, or switches 36A-F. In the illustrative example, button 36A is used to freeze the ECG frame; button 36B is used to transmit the data as explained further herein; button 36C is used to store data; button 36D (FIG. 3) is used to toggle the ECG display between 3-lead, 6-lead, and 12-lead displays; and button 36E (FIG. 3) or 36D (FIG. 8) is used to display patient events window 37 in the ECG and status display areas (FIG. 5). Button 36F allows the user to interface with the AMRD. For example and without limitation, the button 36F may allow a user to access or toggle between information and display screens, to move a cursor within a screen, to enter information and the like. Illustratively, a user could actuate button 36E in order to display the patient events window 37, and then use button 36F to select one or more of the patient events. The device illustratively will record that event or events and the time of entry for correlation with the ECG reading corresponding to that time. Any of the buttons 36A-F could also have different uses or a combination of uses. As seen, for example, in button 36D in FIG. 3 and button 36D in FIG. 8. Also, as seen by comparing button 36F in FIG. 3 with 36F in FIG. 8, the buttons may take on different forms and functioning. The display may also present other data such as for example and without limitation self-diagnostic tests, which may be run automatically or by actuating one of the buttons 36A-F. For example, an impedance test could be run on the leads to ensure that they are placed properly and that they are working properly. In addition to, or instead of the buttons 36A-F, the AMRD may be equipped with another suitable input device, such as a keyboard, a scanner, a bar code reader, a stylus and handwriting recognition system, and/or speech recognition hardware and software to allow a user to interface with the AMRD using voice or other audible commands. As noted, the AMRD illustratively has a speaker or other audible, tactile or visual output device 38. The speaker could give audible commands, information, alerts, or alarms to the user, such as telling the user to take a pill or to lie down, reporting a loose lead or low battery, or providing encouragement. Such information could also or instead be provided on the display, or even by a visible signal, such as a flashing light, or by a tactile alert such as by vibrating or shaking.

In addition to displaying and recording information and data, the AMRD may be equipped with a printer to print the information. Such a printer may be integral to the AMRD or may be linked to the AMRD by one or more signal paths. In addition or instead, as will be described below, the information recorded by the AMRD may be displayed, recorded and/or printed out by the one or more processing units or systems 20 or other computer systems 15. It will be appreciated that the AMRD may be controlled by control circuitry contained within the AMRD itself, or contained within the one or more processing units or systems 20 or other computers 15 in communication with the AMRD. The user may also control the AMRD, manually or through the use of the control circuitry.

The one or more processing units or systems 20, as well as computer systems 15₁ through 15_{M}, each illustratively may comprise a computer 21, which may have an internal memory device 22, and which may be in communication with or networked with one another or with other computer systems 15₁ through 15_{M}. The internal memory 22 may be permanent, semi-permanent, or non-permanent in nature. For example and without limitation the memory 22 may comprise alone or in any combination a chip, a disk, a hard drive, a tape, or a card. Some suitable examples include without limitation random access memory (RAM), read only memory (ROM), video memory card such as Video Random Access memory (VRAM), flash memory, and/or any suitable removable recording media such as a floppy disk, a compact disk (CD), a digital video disk (DVD), or a thumb drive or memory stick. The computer 21, illustratively which may be a super computer, a mainframe computer, a personal computer, a laptop computer, a handheld computer, a personal digital assistant, or the like, may be connected to one or more peripheral devices 23, 25, 27, 29. For example, the computer 21 may be connected via a signal path 24 to an input device 23 such as for example a keyboard. Such a keyboard 23 may be a full-sized keyboard or any other sized keyboard. Other input means and devices may be used, such as for example alone or in combination with the keyboard a scanner, a bar code reader, a stylus and handwriting recognition system, and/or a speech recognition system. The computer 21 illustratively may also be connected via a signal path 26 to a suitable output display device such as for example and without limitation a monitor 25, and/or via signal path 28 to an output display device such as for example printer 27. Printer 27 may be any suitable printer including without limitation a laser printer, an inkjet printer, a thermal printer, or an impact printer. Computer 21 may further be connected via signal path 30 to any external storage device or medium 29. The external storage device 29 may comprise alone or in any combination a chip, a disk, a hard drive, a tape, or a card, including without limitation RAM, ROM, VRAM, flash memory, and/or any suitable removable recording media such as a floppy disk, a CD, a DVD, or a thumb drive or memory stick. The signal paths 24, 26, 28, and 30 illustratively may be two-way and may be any suitable communications pathway or interface such as for example and without limitation the pathway may comprise alone or in combination any suitable hardwire, radio frequency signal, or light signal as has been described herein above with respect to signal paths K and L. Some illustrative connections, mediums, standards, protocols or networks include without limitation, hardwire, USB, FireWire, i.Link, IEEE 1394, ethernet, cable modem, broadband DSL, the Internet, PSTN, telephone modem, micro wave networks, cellular telephone networks, satellite networks, intranets, LAN, WAN, Wireless Area Network or WLAN or any other suitable communication standard, system, interface or protocol such as for example and without limitation any Wi-Fi system or network, including for example and without limitation 802.11a, 802.11b, and 802.11g Wi-Fi systems, bluetooth systems, infrared systems, and the like.

Processing units or system(s) 20 may run any suitable software including commercially available, proprietary or open source software. For example, the computer 21 may run windows-based, Macintosh-based, UNIX-based or Linux-based software as desired. Any software programs may be instructional based and/or use a graphical user interface. The processing system 20 illustratively may use any combination of hardware and software based security and may comply with any applicable industry or governmental standards including without limitation 21 C.F.R. § 11. The system 20 also may support XML and HTML communication and file storage. The processing system 20 may have appropriate database and analysis software, or may use such software resident on one or more of the other computer systems 15. The other computer systems 15 may comprise the same hardware, software, and signal path structure as just described for the one or more processing units or systems 20.

In an illustrative operation 110, a user will initialize 120 each of the plurality of AMRDs by defining, programming, or loading a desired test protocol and/or patient demographics into each AMRD. The test protocol and/or demographics may be loaded directly into each AMRD, or may be loaded into each AMRD through an external computer 15, 21. If the protocol and demographic parameters are loaded into each AMRD using an external computer 15, 21, then such parameters illustratively may be loaded or programmed into the computer 15, 21 using the input device 23 such as for example a keyboard, or the input storage device 29, such as for example uploading from a disk. The protocol may include for example and without limitation the drug or drugs to be tested, the phases of the test, a definition of various symptoms, of the start date and time, of the duration of the test or of the end date and time, of one or more events to be accomplished during the test, and of reasons to end or restart the test, and any other desired parameters. A user may also enter the demographics of subjects or patients participating in the study or test and assign each subject or patient to a unique AMRD, such that each AMRD is associated to one assigned patient only and vice versa. The demographics may include without limitation for each patient the patient's name and identification number and any other information required by the protocol, for example and without limitation the patient's height, weight, current physical condition, current medications, medical history, family history and the like. So too, the patient's personal identification information, such as address and phone number may be entered if desired. Illustratively, the user must enter a security key in order to enter and/or access the demographic information and/or the test results, and an audit trail may be generated for all users who access the information and for any changes made to the information throughout the test.

At any suitable time, the computer 15, 21 and the AMRDs will establish communications via wireless and/or hardwire signal paths as desired, in order to transfer, transmit or communicate the protocol and demographic parameters to each AMRD from the computer 15, 21, or to each computer 15, 21 from each AMRD as appropriate. The transmitted information may be displayed 32 by the AMRD and/or the computer 15, 21. Such display, and or access to the demographics and protocol parameters and information may be controlled by one or more security measures such as for example the USB key 35. It will be appreciated that the AMRDs need not be re-initialized for subsequent tests. For example, the AMRDs could be initialized for and used by a first set of subjects or patients as part of a first test protocol, surrendered by the first set of patients and initialized for and used by a second set of patients as part of a second test using the same or a different protocol, surrendered by the second set of patients and again attached to the first set of patients without the need to reload the protocol and/or demographics for that first set of patients, and so on for however many sets of patients and protocols desired. In other words, each AMRD will retain the data for each test protocol and each subject loaded into that particular AMRD with access controlled by the security key 35, and the desired protocol and demographic information in current use at any one time being controlled by the entry of a patient's unique identification number corresponding to the desired demographic information and the selection of the desired protocol for that patient. Illustratively, an AMRD will retain the protocol and demographic parameters and recorded data until it is purged; therefore, a patient could not only use the AMRD containing that patient's demographic data after that AMRD is used by another subject under a different protocol, but also use that AMRD for a different protocol loaded into the AMRD. So too, the processing system 20 and or other computer 15 illustratively retains the demographic parameters and/or other patient information for entire groups of test patients for recall and uploading in the AMRDs, as well as multiple test protocols for recall and uploading into the AMRDs as desired.

The AMRD may provide an indication of its readiness to start and/or continue a test. For example, the AMRD may conduct a self test and transmit its ready status directly to the processing system 20. The transmission of the foregoing information between the AMRD and the processing system is via any of signal paths L, using any one or combination of the above defined communication means including any suitable hardwire or wireless signal path and connection. In addition, or in lieu of the status communicated to the processing system, the AMRD may itself emit an audible or visual readiness signal or alert indicating among other things that it has sufficient power, and that it has received the protocol and the demographics parameters, including without limitation the symptoms, the desired start time, the desired duration of the test and the identification of the patient associated with that AMRD for that protocol.

The electrodes are attached or connected 130 to the body of a particular subject and to the AMRD assigned to that particular subject. For example, ten electrodes are connected to a subject, and its leads are connected to that subject's uniquely assigned AMRD. As noted above, the connection may be any suitable hardwire or wireless connection. The AMRD illustratively provides a graphical indication 34 of the proper placement and signal quality of the electrodes and leads 119. The AMRD may also provide a textual and/or audible indication of proper placement and signal quality, to include a sounding or displaying an alarm 120 in the event of a signal quality of defined poor quality, and may communicate such status to the one or more processing units 20 via one or more signal path(s) L. The AMRD may be connected to the associated patient at anytime before, after or during the initializing step.

At the defined start time, each of the plurality of AMRDs involved in the test will begin recording simultaneously 140. This simultaneous commencement of recording may be directed by the AMRD's internal software based on the defined start time transmitted from the computer 15, 20 to the AMRD and using a clock internal to the AMRD or using an external clock. For example, the AMRD could receive a clock time from the processing unit 20 via any of the signal paths L, or from any of the other computers 15 directly or through processing unit 20 using any suitable signal path, or even from a global positioning system (GPS) signal from a satellite or some other suitable clock such as an atomic clock. Even if each AMRD has an internal clock, each of those clocks may be synchronized via a signal from an external computer 15, 21 or via a GPS signal and the like. If a particular protocol prescribes different rather than simultaneous start times, then each AMRD will start recording, as just described, as prescribed by the protocol loaded into the AMRD. Illustratively, an alarm or alert may sound and/or be displayed for example and without limitation if the AMRD fails to record, if the electrodes are not functioning properly, if the battery is running low on power, or if the protocol dictates some patient action, such as for example returning to bed.

The text and graphical data recorded by each AMRD is recorded continuously 140 by the AMRD and may be displayed 32, 33, 34 and/or transmitted to an external computer 15, 21 as desired and determined by a user, for example by operation of control buttons 36A-F or other input device or means, by direction of an external computer 15, 21, or by direction of the protocol. In addition to measuring, recording, and displaying ECG data, the AMRD may be equipped to record other physiological data such as for example and without limitation body temperature, pulse rate, blood pressure, FPO₂, respiration, CO₂, and/or brain waves. Also, as noted, a patient may record symptoms experienced by the patient.

The data recorded by the AMRD may be transmitted to the computer 15, 21 using any suitable hardwire or wireless transfer. The transmission may be directed by a user, for example by pushing or actuating button 36b or by executing a command on the computer 15, 21 directing the AMRD to transmit data. The transmission may also be directed by the software in the AMRD or the computer 15, 2, for example and without limitation based on a time certain, based on the elapsing of a certain amount of time, based on the collection of a certain amount of data, or based on the occurrence of a specified event such as an abnormal ECG or the experience and entry by the patient of a certain symptom. If the data is first transferred to one or more other computers 15, then the data may further be transferred to the processing unit 20 for storage 160 and processing 170. Such further transfer, indeed all transfers and transmissions described herein may utilize one or more secure transmission protocols. The data transmitted to the processing unit may be processed or analyzed 170 by a user with software tools, for example an ECG interpretive algorithm, resident on the computer 21, or on another computer 15. Illustratively, the software will eliminate the storage and analysis of duplicate ECGs for each patient, and will not only automatically analyze the data, but also allow a user to review and edit the analysis and to perform a manual analysis. A user may also mine the data with standard and custom queries. Any such review and edit of the data will require authorized access via a security key 35 and will be tracked in accordance with applicable rules, regulations and standards such as for example 21 C.F.R. § 11. For example and without limitation, the date, time, and reason for any changes made by a user will be recorded. The data and the analysis may be stored in one or more data bases, displayed, printed, and/or transmitted or exported to other systems as desired. The storage database(s) may take any suitable form, including without limitation the form specified in Exhibit 1 attached hereto. The data may be converted to XML format for export.

The data recorded by the AMRD may be transmitted to the computer 15, 21 using any suitable hardwire or wireless transfer. The transmission may be directed by a user, for example by pushing or actuating button 36B or by executing a command on the computer 15, 21 directing the AMRD to transmit data. The transmission may also be directed by the software in the AMRD or the computer 15, 2, for example and without limitation based on a time certain, based on the elapsing of a certain amount of time, based on the collection of a certain amount of data, or based on the occurrence of a specified event such as an abnormal ECG or the experience and entry by the patient of a certain symptom. If the data is first transferred to one or more other computers 15, then the data may further be transferred to the processing unit 20 for storage 160 and processing 170. Such further transfer, indeed all transfers and transmissions described herein may utilize one or more secure transmission protocols. The data transmitted to the processing unit may be processed or analyzed 170 by a user with software tools, for example an ECG interpretive algorithm, resident on the computer 21, or on another computer 15. Illustratively, the software will eliminate the storage and analysis of duplicate ECGs for each patient, and will not only automatically analyze the data, but also allow a user to review and edit the analysis and to perform a manual analysis. A user may also mine the data with standard and custom queries. Any such review and edit of the data will require authorized access via a security key 35 and will be tracked in accordance with applicable rules, regulations and standards such as for example 21 C.F.R. § 11. For example and without limitation, the date, time, and reason for any changes made by a user will be recorded. The data and the analysis may be stored in one or more data bases, displayed, printed, and/or transmitted or exported to other systems as desired. The storage database(s) may take any suitable form, including without limitation the form specified in Exhibit 1 attached hereto. The data may be converted to XML format for export.

A user may use the control panel 36, or other control device such as a stylus, a bar code reader or a microphone, to control and interface with the AMRD. For example, as noted above, the user could actuate button 36A (FIGS. 3 and 8) to freeze the ECG frame; button 36B (FIGS. 3 and 8) to transmit data to an external computer 15, 21; button 36C to store data; button 36D to toggle the ECG display between 3-lead (FIGS. 4 and 8), 6-lead (FIG. 7), and 12-lead (FIG. 3) displays; button 36E to display the patient events window 37 (FIG. 5) and button 36F to interface with the AMRD. For example and without limitation, a user could actuate button 36E (FIG. 3) or 36D (FIG. 8) in order to display the patient events window 37, and use button 36F to select one or more of the patient events as appropriate, press button 36C to store the data, press button 36B to transmit the data. The user could also direct that the data be printed, either directly from the AMRD if equipped with a printer, or after the data is transmitted to an external computer 15, 21, the data could be printed on that computer's 15, 21 printer.

While the invention has been illustrated and described in detail in the foregoing drawings and description, the same is to be considered as illustrative and not restrictive in character, it being understood that only illustrative embodiments thereof have been shown and described and that all changes and modifications that come within the spirit of the invention are desired to be protected.

What is claimed is:

1. A method for gathering physiological data from a plurality of patients, the method comprising the steps of:
providing a discrete recording device for each of the plurality of patients;
associating each discrete recording device with its respective assigned patient;
connecting each recording device to its associated patient;
recording onto the recording device physiological data from its respective patient;
transmitting the physiological data between a processing unit and each recording device; and
storing the transmitted data.

2. The method of claim 1 further comprising the step of programming each of the recording devices with one or more parameters.

3. The method claim 2 wherein the programming step includes defining a test protocol.

4. The method of claim 3 wherein the defining a test protocol step includes setting a recording start time.

5. The method of claim 4 wherein each recording device is programmed with and begins the recording step at the same recording start time.

6. The method of claim 4 wherein the defining a test protocol step further includes defining a duration for the test.

7. The method of claim 4 wherein the defining a test protocol step further includes defining one or more times for alerting any one patient to perform some event.

8. The method of claim 7 further comprising the step of alerting any one patient associated with any one of the respective recording devices.

9. The method of claim 8 wherein the alert is provided simultaneously to each of the patients.

10. The method of claim 8 wherein the respective recording device provides the alert.

11. The method of claim 2 wherein the programming step further includes defining one or more times certain for a scheduled transmission of physiological data from the recording device to the processing unit during the transmitting step or steps.

12. The method of claim 3 wherein the defining a test protocol step further includes defining one or more physiological symptoms of interest.

13. The method of claim 12 wherein the defining a test protocol step includes defining which of the one or more physiological symptoms of interest will signal an unscheduled transmission of physiological data from the recording device to the processing unit.

14. The method of claim 13 wherein the patient initiates the transmitting step in response to one of the symptoms of interest.

15. The method of claim 2 wherein the programming step further includes entering patient demographics.

16. The method of claim 15 wherein the entering patient demographics step includes entering the demographics information of each of the plurality of patients.

17. The method of claim 2 wherein the programming step further includes specifying the resolution of a medical measuring device in communication with a respective recording device and used to measure one or more physiological parameters that make up the physiological data.

18. The method of claim 17 wherein the medical measuring device comprises an electrocardiogram (ECG) device coupled to the recording device and wherein the specifying step further includes specifying the resolution of the ECG.

19. The method of claim 2 further comprising the step of providing secure access to the recording device.

20. The method of claim 19 wherein the providing secure access step includes using an authentication device to control access.

21. The method of claim 2 further including the step of transmitting to the recording device the parameters entered into the processing unit during the programming step.

22. The method of claim 2 wherein each processing unit and each recording device can both transmit and receive signals between one another.

23. The method of claim 22 wherein the signals transmitted between the recording device(s) and the processing unit are transmitted using any suitable wireless or hardwire transmission technique.

24. The method of claim 23 wherein the processing unit comprises a network of processing units.

25. The method of claim 2 wherein the connecting step includes connecting to each respective patient one or more monitoring electrodes of the ECG device.

26. The method of claim 25 further comprising the step of determining the status of the recording device.

27. The method claim 26 wherein the determining the status step comprises:
transmitting parameters from the processing unit to each recording device;
evaluating the readiness of each respective recording device;
determining the signal quality of the electrodes;
providing an alarm if an abnormal condition is detected in the recording device, the ECG device, the electrodes, and/or communications between any of the processing unit, the recording device, the ECG device and the electrodes; and
transmitting from each recording device to the processing unit a ready to proceed signal.

28. The method of claim 27 further comprising the step of displaying on a display screen of the recording device certain of the parameters transmitted from the processing unit, wherein the displayed parameters may include for a respective recording device its associated patient's name, its patient's identification number, the protocol being used, medication administered to the patient, current date, current time, and a symptom list.

29. The method of claim 25 wherein the recording step comprises the steps of:
entering patient data into the recording device;
reading continuous ECG clinical data received from the electrodes;
converting an analog signal from the electrodes to a digital signal;
displaying on the recording device the ECG data; and
storing for a minimum of 24 hours the entered patient data and the ECG data in any suitable storage medium.

30. The method of claim 2 wherein the transmitting step comprises transmitting between the recording device and the processing unit one or more of the following: physiological data, patient events information, interaction logs, performance status of the recording device, performance status of the processing unit, transmission schedules or any other data recorded, stored or displayed on either the recording device or the processing unit.

31. The method of claim 2 further comprising the step of directing at a predetermined time defined by the protocol the patient to perform an event.

32. The method of claim 2 wherein the storing step includes storing on any suitable medium coupled to the processing unit the data transmitted from the recording device.

33. The method of claim 32 further comprising the step of analyzing the stored data which includes the steps of:
retrieving the stored data;
viewing the retrieved data on an output device;
selecting the data to be analyzed; and
performing the analysis of the selected data.

34. The method of claim 33 wherein the analysis performing step comprises taking manual measurements of the ECGs.

35. The method of claim 33 wherein the analysis performing step comprises applying an automated interpretive algorithm to the selected data.

36. The method of claim 33 wherein the analysis performing step comprises:
converting the data to XML format; and
exporting the converted data to a third-party processing unit for analysis by an automated third-party algorithm.

37. The method of claim 33 wherein the analyzing step further comprises the steps of:
generating a file of unconfirmed ECGs not associated with a patient;
confirming the unconfirmed ECGs by collating them with patient entered data; and
performing the analysis on the confirmed ECGs.

38. The method of claim 33 further comprising the steps of:
editing the analysis;
acting on the analysis as defined by the protocol; and
storing the results of the analysis.

39. The method of claim 38 further comprising the steps of:
retrieving the stored data and/or the stored analysis;
mining the stored data and/or the stored analysis;
transmitting the stored data and/or analysis to one or more third-party.

40. The method of claim 2 wherein the method is compliant with defined government standards.

41. The method of claim 40 wherein the defined standard is 21 C.F.R. § 11 extant on Dec. 20, 2005.

42. A system for gathering physiological data from a plurality of patients, the system comprising:
a discrete recording device associated with each of a plurality of patients;
a sensor coupled to each recording device, the sensor configured to gather physiological data from the patient; and
a processing unit in two-way communication with each of the recording devices;
wherein each discrete recording device is configured to continuously receive, store, display, analyze and transmit physiological data gathered from its associated patient; and
wherein the processing unit is configured to receive, store, output, and analyze the data transmitted by each recording device.

43. The system of claim 42 wherein each discrete recording device and the processing unit each comprises one or more displays capable of displaying the physiological data in a user selectable format.

44. The system of claim 43 wherein the physiological data includes ECG data and the one or more displays is capable of displaying the data in a user selectable format chosen from the list comprising 3-lead, 6-lead, 12-lead and freeze formats.

45. The system of claim 43 further comprising storage media capable of continuous recording of ECG data for at least about 24 hours.

46. The system of claim 43 wherein each recording device further comprises:
an analog to digital converter;
a clock;
control software;
a diagnostic system;
an alarm system;
one or more control keys; and
a security system.

47. The system of claim 46 wherein the alarm system is responsive to an abnormal condition determined by the self-test algorithm.

48. The system of claim 46 wherein the alarm system is responsive to the occurrence of a predefined event.

49. The system of claim 46 wherein each display is configured to display certain patient data including the patient's name, the patient's identification number, the patient's medicine, the patient's test protocol, the current date, and the current time.

50. The system of claim 46 wherein the security system controls access to the system.

51. The system of claim 46 wherein the sensor comprises one or more electrodes coupled each recording device.

52. The system of claim 46 wherein the resolution of each sensor is adjustable.

53. The system of claim 46 further comprising one or more peripheral devices coupled to the processing unit.

54. The system of claim 53 wherein the one or more peripheral devices is an output device selected from the group consisting of a display, a printer, and a storage device.

55. The system of claim 54 wherein the printer is an impact printer, a laser printer or an inkjet printer.

56. The system of claim 53 wherein the one or more peripheral devices is an input device selected from the group consisting of a keyboard, a scanner, and a mouse.

57. The system of claim 46 wherein each recording device and the processing unit are coupled together by any suitable means.

58. The system of claim 46 wherein each recording device and the processing unit are coupled together by hardwire.

59. The system of claim 46 wherein each recording device and the processing unit are coupled together by a wireless communications network.

60. The system of claim 43 wherein the processing unit comprising means for:
entering protocols;
synchronizing the clocks of the processing unit and each recording device;
assigning each discrete recording device to each patient;
inputting patient data;
providing secure access control;
determining transmission schedules;
providing two-way communications with each recording device;
analyzing gathered and inputted data;
storing the analysis; and
exporting the analysis and the data.

61. The system of claim 43 further comprising one or more additional processing units networked together with said first processing unit.

62. The system of claim 43 wherein the entire system is 21 CFR § 11 compliant.

63. A system for gathering physiological data from a plurality of patients, the system comprising:
a discrete recording device associated with each of a plurality of patients, each recording device including:
an analog to digital converter;
a clock;
control software;
a diagnostic system;
an alarm system;
a display;
one or more control keys; and
a security system
a sensor coupled to each recording device, the sensor comprising one or more electrodes configured to gather physiological data from the patient; and
a processing unit in two-way communication with each of the recording devices, the processing unit including
a processing unit clock;
processing unit control and analysis software;
a processing unit diagnostic system;
a processing unit display;
a processing unit printer; and
a processing unit security system;
wherein each discrete recording device is configured to continuously receive, store, display, analyze and transmit physiological data gathered from its associated patient; and
wherein the processing unit is configured to receive, store, output, and analyze the data transmitted by each recording device.
